(19) United States
(12) Patent Application Publication
(10) Pub. No.: US 2012/0071962 A1
(43) Pub. Date: Mar. 22, 2012

(54) INTRAVASCULAR STENT

(75) Inventors: Howard Huang, Santa Clara, CA (US); John Yan, Los Gatos, CA (US); Vinayak Bhat, Cupertino, CA (US); Motasim Sirhan, Los Altos, CA (US)

(73) Assignee: Elixir Medical Corporation, Sunnyvale, CA (US)

(21) Appl. No.: 12/965,080

(22) Filed: Dec. 10, 2010

Related U.S. Application Data
(63) Continuation of application No. PCT/US2009/047105, filed on Jun. 11, 2009.
(60) Provisional application No. 61/060,994, filed on Jun. 12, 2008.

(51) Int. Cl. A61F 2/82

(52) U.S. Cl. 623/1.16; 623/1.46; 623/1.42

(57) ABSTRACT

Stents are provided with scaffold structures which have low exposures when implanted in arteries and other blood vessels and lumens. The cross-sectional dimensions, materials, and patterns are controlled to provide sufficient strength and coverage while maintaining the low exposure.

### CROSS-REFERENCES TO RELATED APPLICATIONS

The present application is a continuation of International Patent Application No. PCT/US2009/047105 (Attorney Docket No. 022265-000710PC), filed Jun. 11, 2009, which claims priority from U.S. Provisional Patent Application No. 61/060,994 (Attorney Docket No. 022265-000700US), filed Jun. 12, 2008, the full disclosures of which are incorporated herein by reference.

### BACKGROUND OF THE INVENTION

### Field of the Invention

The invention relates to vascular repair devices, and in particular intravascular stents which are adapted to be implanted into a patient's body lumen, such as a blood vessel or coronary artery, to maintain the patency thereof. Stents are particularly useful in the treatment of atherosclerotic stenosis in arteries and blood vessels.

Biomedical stents are generally tubular-shaped devices which function to hold open or reinforce a segment of an artery, vein, or other body lumen, such as a coronary artery, a carotid artery, a saphenous vein graft, a femoral artery, a ureter, vein grafts, and the like. They also are suitable for use to support and hold back a dissected arterial lining that can occlude the fluid passageway, stabilize plaque, or to support bioprosthetic valves.

At present, there are numerous stent designs. For example, a prior art stent 10 depicted in FIG. 1 includes a number of cylindrical rings, i.e. each including struts 14 connected by crowns 16. The rings 12 may be joined directly (crown to crown) but will more typically be joined by links or connectors 18 which may be linear (as illustrated), S-shaped, M-shaped or the like. Most presently available coronary stents typically have struts that range in width from 0.0035 inch to 0.0050 inch and in thickness 0.0023 inch to 0.0060 inch.

In addition to strut dimensions, stents may be characterized by other known parameters such as metal-to-artery ratio, which is the ratio of the outer surface area of the stent to the area of the vessel wall being stented at the expanded diameter of the stent, typically expressed as a percentage and ranging from 12 to 20%. Examples of metal-to-artery ratios include the ACS Multi-Link® stent, which is 15% at 3 mm diameter and the Medtronic Driver® stent, which is 19% at 3 mm diameter.

Stents can be delivered to the target area within the body lumen using a catheter. With a balloon-expandable catheter the stent is mounted onto the balloon and navigated to the appropriate area, and the stent expanded by inflating the balloon. A self-expanding stent is delivered to the target area and released from constraint to deploy to the required diameter.

What has been needed and heretofore unavailable is a stent design which aids in healing of the vessel and/or endothelialization/cellularization of the stent, and/or causes less injury to the vessel, and/or minimizes foreign body material in the vessel. The present invention satisfies at least some of this need.

### SUMMARY OF THE INVENTION

The present invention presents an implantable stent or prosthesis that is used for treating vascular conditions.

The stent of the present invention has low luminally exposed surface area and presents less foreign body material within a vessel. Furthermore, the stent's structural members, which include struts, are configured to facilitate reduction of the exposed surface area of the stent. The structure of the stents are usually formed from a scaffold comprising a series of connected rings, each including struts and crowns. The rings are typically connected by connectors (links) but in some cases crowns may be connected directly to adjacent crowns to form the body of the stent. The scaffolds will usually be balloon expandable, more usually being formed from a malleable metal. The metal scaffold, however, may be coated, covered, laminated with or otherwise joined to polymeric and other non-metallic materials. The scaffold may have an open cell structure, a closed cell structure, or a combination of both. Open and closed cell structures are well known and described in the patent and medical literature.

In one embodiment, at least some of the stent strut widths (measured in a circumferential direction) are designed to range from 0.0004 inch to 0.0035 inch, preferably 0.0005 inch to 0.003 inch, most preferably 0.0005 inch to 0.0027 inch. Such narrow widths reduce the exposed surface area of the stent.

In another embodiment of the present invention, at least some of the stent strut thicknesses (measured in a radial direction) are designed to be in the range of 0.001 inch to 0.005 inch, more preferably 0.001 inch to 0.0032 inch, and most preferably 0.001 inch to 0.0025 inch. Such thicknesses also reduce the exposed surface area of the stent, particularly when combined with the widths above.

In another embodiment of the present invention, at least some of the stent strut lengths (measured from peak to peak along the elongate axis of the strut) are designed to be in the range of 0.1 mm to 2 mm, more preferably 0.2 mm to 1.5 mm, most preferably 0.2 mm to 1 mm. Such length also helps reduce the exposed area of the stent, particularly when combined with the widths and thicknesses above. This helps designing a stent with less foreign body material. It may also help designing a stent to have adequate coverage of the vessel and yet have some recoil especially when combined with strut width, thickness as described in the present invention.

Preferably, the stent scaffold dimensions including width and thickness will be selected to provide a cross-sectional area of the strut in the range from 1 to 6 mils², preferably from 1.5 to 5 mils², most preferably from 2 to 4 mils² (one mil²=0.000001 inch). The stent strut cross-sectional geometries may be rectangular, square, tapered, or irregular, usually being rectangular or tapered with the thickness being greater than the width.

In another embodiment of the present invention the stent is configured to have a metal-to-artery ratio ranging from 1% to 12%, preferably 2% to 9%, most preferably 3% to 7%, when measured at the nominal (labeled) expanded diameter of the stent. Typical expanded diameters range from 1.5 mm to 25 mm, more preferably 2 mm to 4 mm.

In another embodiment of the present invention the scaffold of the stent is configured to have a maximum total stent cross-sectional area of less than 100 mils², preferably less than 75 mils², most preferably less than 50 mils². This is the total cross-sectional area of the stent scaffold when the stent is cut diametrically along the length which would give the maximum cross-sectional area. The area includes the cross-sections of the struts, links, connectors and any other structures, usually structural scaffold material(s) which may be present (excluding coatings, graft coverings, and other components which are not part of the metal scaffold.)

In another embodiment of the present invention the scaffold of the stent is configured to have an outer surface area of less than 0.8 mm² per mm stent length, preferably less than 0.6 mm² per mm scaffold length, most preferably less than 0.4 mm² per mm scaffold length. This is the surface area of the outward-facing surface of the scaffold per unit length of the stent when expanded to its nominal (labeled) diameter stent.

In another embodiment of the present invention the scaffold of the stent is configured to have a total stent surface area of 1 mm² to 3.7 mm² per mm stent length and preferably 1 mm² to 3 mm² per mm stent length when expanded to the nominal (labeled) diameter. In this embodiment the surface area includes all exposed surfaces (i.e., inside surfaces, outside surfaces, and radially aligned surfaces) but excludes holes, indentations or texturing-effects that maybe present on the stent surface.

In another embodiment of the present invention the scaffold of the stent is configured to have a total volume of 0.008 mm³ to 0.06 mm³ per mm of length, preferably 0.02 mm³ to 0.05 mm³ per mm of length when expanded to the nominal (labeled) diameter. The total scaffold volume is the total volume of metal or other structural scaffold material in the stent and may be calculated by multiplying the values above by the length of the scaffold in mm.

In another embodiment of the present invention the stent scaffold is configured to have a maximum circular unsupported area of less than or equal to 2.0 mm diameter (3.14 mm² area), preferably less than or equal to 1.5 mm diameter (1.77 mm² area), most preferably less than or equal to 1.0 mm diameter (0.8 mm² area). This is the largest circular gap between struts present when the stent is expanded to its nominal (labeled) diameter. This provides for adequate scaffolding of the vessel especially when combined with strut widths and thicknesses and/or lengths as described in the present invention.

In another embodiment of the present invention, the stent scaffold is configured to have 2 crowns to 12 crowns per ring, preferably 3 crowns to 10 crowns per ring, more preferably 4 crowns to 8 crowns per ring. In another embodiment of the present invention, the stent is configured to have 4 struts to 22 struts per ring, preferably 6 struts to 20 struts per ring, more preferably 8 struts to 16 struts per ring. In a preferred embodiment, a stent having 6 to 12 crowns can provide adequate scaffolding of the vessel especially when combined with strut widths and thicknesses and/or strut length as described in the present invention. This may also facilitate adequate stent function such as recoil as described in the present invention.

In another embodiment of the present invention, the stent scaffold is configured to have features which facilitate embedding of the stent into the vessel wall upon expansion of the stent.

In another embodiment of the present invention, the stent scaffold is formed from cobalt-chromium L-605 and is configured to have a weight less than 1 mg per mm length, preferably less than 0.5 mg per mm length, and most preferably less than 0.25 mg per mm length. The mass for other metals/alloys will be adjusted by the material density of the stent scaffold and would weigh proportionally relatively more or less than when designed from cobalt-chromium L-605. For example, a 316L stainless steel stent would weigh approximately 13% less since stainless steel has approximately 13% lower density than cobalt-chromium L-605 and therefore a stent designed from 316L would be configured to weigh less than 0.87 mg per mm length, preferably less than 0.44 mg per mm length, and most preferably less than 0.22 mg per mm length.

In another embodiment of the present invention, the stent is implanted so that at least some of the struts and other components of the scaffold at least in part embed into the vessel wall upon expansion of the stent. The amount of embedding can be 20% to 100%, preferably 50% to 100%, most preferably 70% to 100%. The struts may embed completely below the surface of the vessel.

In another embodiment of the present invention, the stent scaffold is implanted to have a maximum ratio of cross-sectional metal to vessel lumen area less than 1.1%, preferably less than 0.9%, most preferably less than 0.5%. This is the ratio of the maximum total cross-sectional area of the stent taken along any point on its length compared to the cross-sectional area of the vessel lumen at that point.

In another embodiment of the present invention, the cross-sectional geometry of the strut is designed to facilitate reducing the exposed surface area of the stent scaffold. The cross-section may be shaped like a square, a rectangle, a triangle, a pentagon, a diamond, a teardrop, a symmetrical or asymmetrical right angle triangle, or other variations. The cross-sectional geometry can influence how the strut interacts with the vessel wall. By selecting the strut width and/or cross-sectional geometry of the stent strut, controlling the amount the stent strut embedding into the vessel wall is facilitated.

In another embodiment of the present invention, the cross-sectional geometry of the strut is selected to present a smaller area exposed to the lumen, such as an inverted triangle, diamond shape, or any shape that has a narrow/small width portion of the strut exposed to the vessel lumen. Preferably, the width of the exposed inner surface of the stent strut ranges from 0.0004 inch to 0.0027 inch.

The stents or other prostheses of the present invention may be deployed using a delivery device with an expandable member, such as a balloon or mechanical spring. The expandable member, such as a balloon, will usually provide an expansion pressure sufficient to fully deploy the stent. In a preferred embodiment, the expansion pressure is usually greater than 5 atm, more preferably greater than 10 atm, most preferably greater than 12 atm. Alternatively, the stents may be constrained by a sheath or other physical means, and deploy by self-expanding when the constraint is removed or activated. The constraint may be removed or activated through physical means, electrical currents, magnetic fields, or application of heat, or other means.

The stent may or may not require pre-dilation and/or post-dilatation of the vessel.

The stent scaffolds of the present invention in preferred embodiments will usually be configured to have at least one or more additional characteristics or properties which provide sufficient strength and/or performance characteristics for the small sized scaffolds which are utilized. Usually, at least one ring or other structural component of the stent of the present invention will have a strength (crush strength) of at least 2 psi to crush at least 25% in diameter, preferably at least 6 psi to crush at least 25% in diameter, and most preferably at least 8 psi to crush at least 25% in diameter. In many embodiments, the entire scaffold of the stent will have a minimum strength (crush strength) and as just set forth. Specific protocols for measuring the strength (crush strength) are described below. The crush strength will typically be no more than 15 psi to crush at least 25% in diameter, preferably no more than 12 psi to crush at least 25% in diameter, and most preferably no more than 10 psi to crush at least 25% in diameter. Preferred strength (crush strength) ranges are 2-15 psi to crush at least 25% in diameter, preferably 2-12 psi to crush at least 25% in diameter, and most preferably 2 to 10 psi to crush at least 25% in diameter. These ranges conform closer to the native vessel compliance and therefore may reduce trauma/injury to the vessel.

The stent and stent scaffold in a preferred embodiment may also be configured to have an acute recoil less than 15%, preferably less than 10%, and most preferably less than 5%. Methods for measuring acute recoil are described below. In another preferred embodiment, the scaffold is configured to have some acute recoil to minimize injury of the vessel. This can be achieved by increasing the number of crowns in a scaffold and/or having strut width, thickness, and/or length as described in the present invention. Preferred acute recoil ranges from greater than or equal to 1% and less than 15%, preferably greater than or equal to 1% and less than 10%, and most preferably greater than or equal to 1% and less than 5%.

The stent and stent scaffold in a preferred embodiment will typically be configured to have any combination of at least one or more of the aforementioned characteristics and in addition be adapted to foreshorten in length by less than 20%, preferably less than 15%, and most preferably less than 10%, when expanded to its nominal (labeled) diameter.

In another embodiment of the present invention, it is desirable to have a low crimped stent profile in order to facilitate easier access to the target site, in this embodiment a stent scaffold is configured to have any combination of at least one or more of the embodiments and has a crimped profile of less than 0.045 inches, preferably less than 0.040 inches, most preferably less than 0.030 inches.

In another embodiment of the present invention, a stent scaffold is configured to have any combination of at least one or more of the embodiments and has a crimped profile ranging from 0.01 to 0.045 inches, preferably ranging from 0.01 to 0.04 inches, most preferably ranging from 0.01 to 0.03 inches.

The stent scaffold may be configured to have a modified surface to promote adhesion of drugs, pharmacological agents, and/or coatings. The modified surface can be achieved through use of various methods, including microblasting, laser ablation, chemical etching, or imparting an ionic or magnetic charge to the surface or any other means.

Stents and stent scaffolds incorporating one or more of the features described above are able to provide healing of the vessel, cellularization/endothelialization of the stent, and/or less injury to the vessel faster than a stent without incorporating one or more of the embodiments of the present invention.

In another embodiment of the present invention, the exemplary and preferred characteristics of the present invention result in more healing of the vessel, more cellularization/endothelialization of the stent, and/or less injury to the vessel, and/or less foreign body material in the vessel than a stent lacking these characteristics. For example, implantation of the stents of the present invention may result in less thrombus formation as a result of more endothelialization than a stent without incorporating one or more of the embodiments of the present invention. The stents of the present invention may also require reduced medication and/or reduced duration of medication to be administered after the implantation procedure.

The stent can be formed using various manufacturing techniques, including injection molding, laser cutting, wire bending, welding, chemical etching, and metal deposition, followed in some cases by descaling, bead blasting, and electropolishing as necessary depending on the material and process used. The stent can be formed from metals including alloys, polymers, ceramics, or combinations thereof, examples include stainless steel alloys, steel alloys, cobalt-chromium alloys, nickel-titanium alloys, platinum-iridium alloys, platinum enhanced alloys such as PERSS (Platinum Enhanced Radiopaque Stainless Steel), molybdenum-rhenium alloys such as NULOY™, magnesium alloys, Elgiloy, platinum, tantalum, titanium, iron, niobium, magnesium, palladium, PLLA, PLGA, etc.

The stent may also elute various drugs/pharmacological agents to reduce tissue inflammation, restenosis or thrombosis and/or to promote healing and biocompatibility of the vessel or stent. In addition the surface of the stent may or may not be covered with a coating such as a polymer. The surface may further be bioactive, including the use of endothelial progenitor cells (EPC) or cell specific peptide linkers. The stent may be permanent or removable, degradable or non-degradable or partially degradable. Additionally, the structure of the stent may be fully or partially covered by membrane/elements on the inside or outside of the stent to provide increased stent coverage.

In one embodiment, the stent is coated at least in part with polymers. The polymers may be non-erodable/non-degradable or bioerodible/biodegradable coatings. Suitable non-erodable/degradable or slow degrading coatings include, but are not limited to, polyurethane, polyethylenes imine, ethylene-vinyl acetate copolymers, ethylene vinyl alcohol copolymer, polyvinylidene fluoride, polyvinylidene fluoride-co-hexafluoropropylene, polytetrafluoroethylene (PTFE), fluropolymers (e.g., PFA, FEP, ETFE, or others), polyvinyl ethers such as polyvinyl methyl ethers, polystyrenes, styrene-maleic anhydride copolymers, polystyrene, polystyrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-isobutylene-styrene block copolymers), silicone, C-flex, nylons, polyamide, polyimide, parylene, parylast, polymethyl methacrylate butyrate, poly-N-butyl methacrylate, polybutyl methacrylate copolymer with polyethylene vinyl acetate (e.g. Polyhexyl methacrylate-co-vinyl pyrrolidinone-co-vinyl acetate and polybutyl methacrylate-co-vinyl acetate), polymethyl methacrylate, phosphorylcholine, poly 2-hydroxy ethyl methacrylate, polyisobutylene, poly ethylene glycols, poly ethylene glycol methacrylates, poly vinyl chloride, polydimethyl siloxane, polytetrafluoroethylene, polyethylene oxide, poly ethylene vinyl acetate, poly carbonate, poly acrylamide gels, poly-N-vinyl-2-pyrrolidone, polyvinyl pyrrolidinone, poly maleic anhydride, quarternary ammonium compounds including stearyl ammonium chloride and benzyl ammonium chloride, and the like, including other synthetic or natural polymeric substances; mixtures, copolymers, or combinations thereof. The coating can be a blend, layering, or copolymer of two or more of these or other polymers. The coating can also be nanostructured coating made from stainless steel, tantalum, or the like.

Suitable bioerodable/biodegradable coatings include, but are not limited to, polylactic acid, polylactides, poly lactates, hydroxyacid polylactic acid polymer, polyglycolic acid, polyglycolates and copolymers and isomers, polydioxanone, polyethyl glutamate, polyhydroxybutyrate, polyhydroxyvalerate and copolymers, polycaprolactone, polyanhydride, polyortho esters, polyether esters, polyiminocarbonates, starch based polymers, polyester amides, polyester amines, Hydroxyapatite, cellulose acetate butyrate (CAB), cellulose, cellulose analogs (e.g., hydroxyethyl cellulose, Ethyl cellulose, Cellulose propionate, cellulose acetate), collagen, elastin, polysaccharides, hyaluronic acid, sodium hyaluronic Acid, hyaluronan hyaluronate, non-sulfated glycosaminoglycan, polycyanoacrylates, polyphosphazenes, copolymers and other aliphatic polyesters, or suitable copolymers thereof including copolymers of poly-L-lactic acid and poly-e-caprolactone; mixtures, copolymers, or combinations thereof.

In one embodiment, the stent may be coated at least in part with at least one pharmacological agent, such as immunomodulator macrocyclic lactones, anti-cancer, anti-proliferative, anti-inflammatory, antithrombotic, antiplatelet, antifungal, antidiabetic, antihyperlipidimia, antiangiogenic, angiogenic, antihypertensive, healing promoting drugs, or other therapeutic classes of drugs or combination thereof. Illustrative pharmacological agents include but are not limited to macrocyclic lactones such as rapamycin, everolimus, Novolimus, ABT 578, AP20840, AP23841, AP23573, CCI-779, deuterated rapamycin, TAFA93, tacrolimus, cyclosporine, TKB662, anti-proliferatives such as as taxol, anti-inflammatories such as dexamethasone, anti-platelet such as trapidil, their analogues, pro-drug, metabolites, slats, or others or combination thereof, antithrombotic such as heparin, analogues, pro-drugs, metabolites, salts, etc. These agents can be coated on the stent surface, mixed with a polymer as a matrix, coated adjacent to a polymer barrier, or covalently or ionically bonded to the polymer.

In order to promote increased adhesion of drugs, pharmacological agents and/or coatings to the stent, the surface of the stent may be modified or treated. This includes microblasting, laser ablation or contouring, chemical etching, or imparting an ionic or magnetic charge to the surface or other means of modifying the stent surface.

In one embodiment, incorporating one or more embodiments of the present invention provides for lower luminal exposed surface area and presents less foreign body material within a vessel compared to stents without the one or more embodiments. Furthermore, the stent's struts are designed to facilitate reduction of the exposed surface area of at least portion of the stent structure.

The accompanying drawings illustrate some embodiments of the present invention, but do not limit the invention to these specific drawings/embodiments. The figures are merely illustrative, not drawn to scale, and not limiting of the many possible implementations of the invention.

### BRIEF DESCRIPTION OF THE DRAWINGS

FIG. 1 illustrates a prior-art stent design in the un-expanded state having multiple rings, formed from crowns and struts. The rings are connected to each other by links. The struts are 0.004 inch wide, the metal-to-artery ratio is 17% at 3 mm diameter, and the total stent surface area is 107 mm² for an 18.9 mm stent (5.7 mm² per mm stent length).

FIG. 2 illustrate a representative prior-art stent expanded inside the lumen drawn in cross-section.

FIG. 3 illustrates a stent pattern comprising a plurality of rings 20 including struts 22 and crowns 24 joined by axial connectors 26.

FIG. 4 illustrates a stent strut cross section with strut width ranging from 0.0004 inch to 0.0027 inch wide, and multiple examples of strut cross-section geometries.

FIG. 5a illustrates the present invention expanded inside the lumen.

FIG. 5b illustrates the present invention with a square strut cross-section, expanded inside the lumen.

FIG. 5c illustrates the present invention with rectangular strut cross-section, expanded inside the lumen.

FIG. 5d illustrates the present invention with inverted triangular strut cross-section, expanded inside the lumen.

FIG. 5e illustrates the present invention with inverted triangular strut cross-section, expanded inside the lumen.

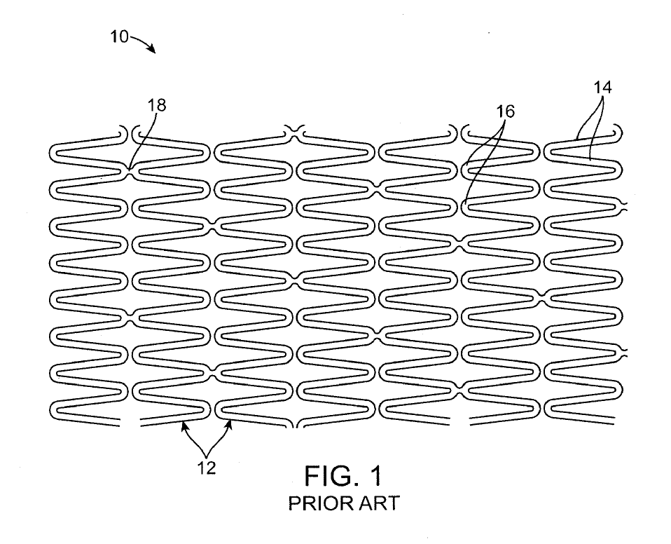

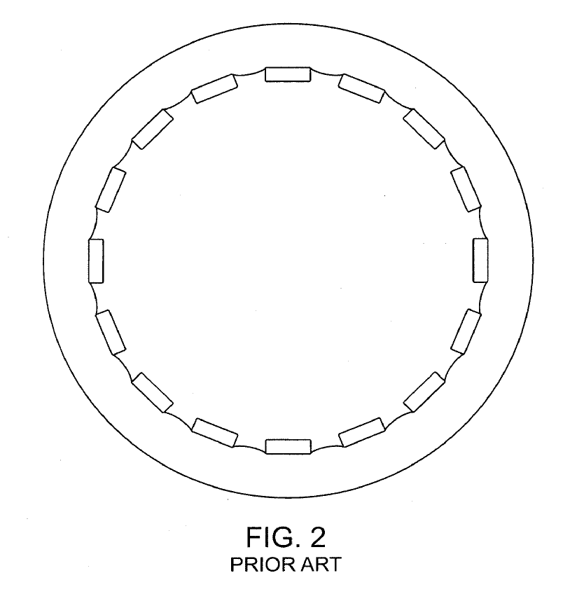

### EXAMPLES

### Example 1

An expandable stent design in the un-expanded state with multiple rings, formed by crowns and struts in a generally undulating pattern, joined by connectors (links) or bridges, with struts 0.0015 inch wide and 0.0032 inch thick. The stent was mounted onto a balloon catheter, crimped, and expanded to the intended diameter (labeled diameter example 3.0 mm). The stent had 5% metal-to-artery ratio at 3 mm diameter. The total stent surface area is approximately 3.1 mm2 per mm stent length and the total stent volume is 0.041 mm3 per mm stent length. The stent is designed utilizing L-605 cobalt-chromium material. The expanded stent was tested for strength (crush strength). The crush strength was measured to be 10 psi when crushed at least 25% and recoil was measured to be 3%. The maximum total stent cross sectional area of the stent is 72 mil squared.

### Example 2

In another example, a stent was designed utilizing 1045 carbon steel. The carbon steel stock was drawn into 0.0625 inch diameter hypotubes. Stents were laser cut using a pulsed-laser CNC machine (LPL Systems, Mountain View, Calif.), and polished to the nominal dimensions using an electropolishing station filled with steel polishing solution (ESMA Inc, South Holland, Ind.) at the appropriate time and current settings, then coated with a PLGA polymer/macrocyclic lactone drug matrix, and mounted onto 3 mm and 3.5 mm diameter balloon catheters. The 5 crown stent was designed to nominal dimensions of 0.002 inch wide×0.0024 inch thick, with a cross-sectional strut area of 4.8 mils² and a maximum total stent cross-sectional area of 48 mils². The metal-to-artery ratio was designed to be 5-6% at 3 mm diameter. The total stent surface area was approximately 2.3 mm² per mm stent length and the stent volume is 0.033 mm³ per mm stent length. In vivo studies utilizing this stent were conducted for 28 and 90 days in 10-16 week old young adult Landrace-Yorkshire hybrid farm pigs, a non-atherosclerotic swine model chosen because the model has been used extensively for stent and angioplasty studies, resulting in a large volume of data on its vascular response and correlation to humans. The appropriately sized stent was introduced into the artery by advancing the stent mounted balloon catheter through the guide catheter and over the guidewire to the deployment site. The balloon was then inflated at a steady rate to a pressure sufficient to target a balloon to artery ratio of 1.1:1.0, at pressures up to 20 ATM. At the designated timepoint, animals were tranquilized and anesthesized and angiograms were recorded. Each implanted stent was quantitatively evaluated for lumen narrowing using quantitative coronary angiography (QCA). All vessels were patent after 28 and 90 days. Some of the stents were expanded on the bench to (to labeled diameter of 3.0 mm). The stent strength (crush strength) was measured to be 8 psi when crushed at least 25% in diameter using a clamshell radial crush strength test method. In a clamshell radial crush strength test method, the stent (or part of the stent) is expanded in air to its intended diameter (for example, 3 mm labeled diameter) and placed inside a set of blocks with grooves (semi-circular in shape). The blocks are mounted in a force-displacement test machine, such as an Instron 5540 series materials testing system, and the stent (or part of the stent) is crushed 25% in diameter. The force is converted to force per unit area (i.e. PSI) by dividing the peak load by the longitudinal cross-sectional area of the stent (or part of the stent). Alternatively, another test to measure the strength (crush strength) is the pressure vessel test. In a pressure vessel test, the stent is expanded into a tube to its intended diameter (labeled diameter). The tube is then placed in a pressure vessel. The pressure vessel is pressurized, pressurizing the tube, until the stent or part of the stent is crushed at least 25% in diameter. A pressure gauge records the output pressure reading in PSI. The stent recoil was measured to be <5%. Recoil was measured on a optical comparator, such as a Micro-Vu Precision Measuring System (Micro-Vu Corp, Windsor, Calif.). Recoil was characterized by inflating the stent on balloon to the nominal intended diameter, and then measuring and recording the average initial stent diameter, then deflating the balloon, re-measuring the average stent final diameter, and dividing the change in diameter by the initial diameter. Multiplying by 100 yields the percent recoil.

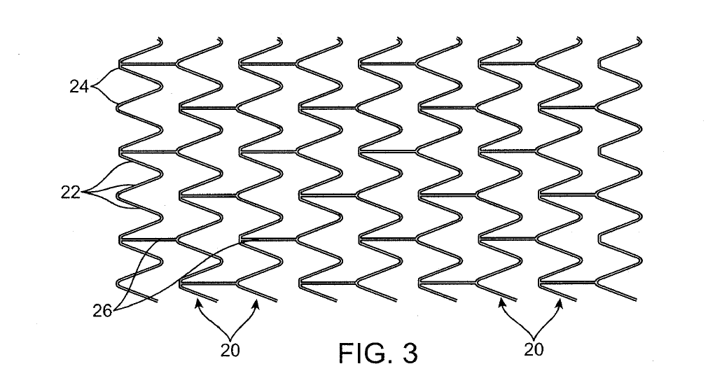

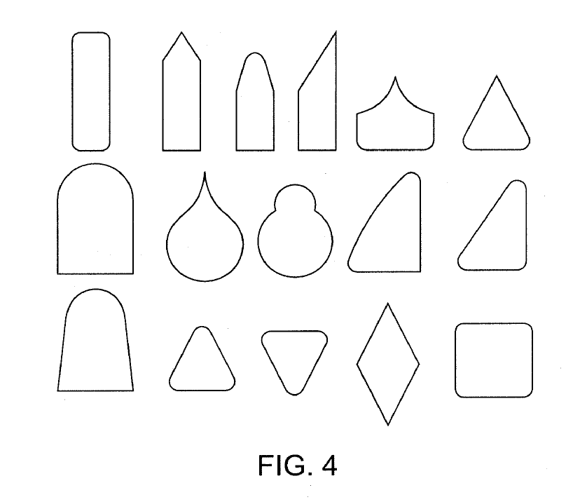

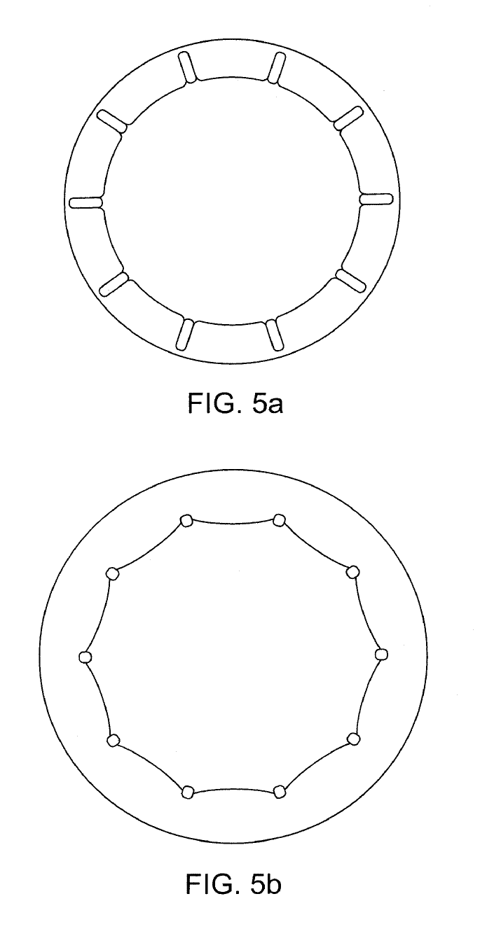

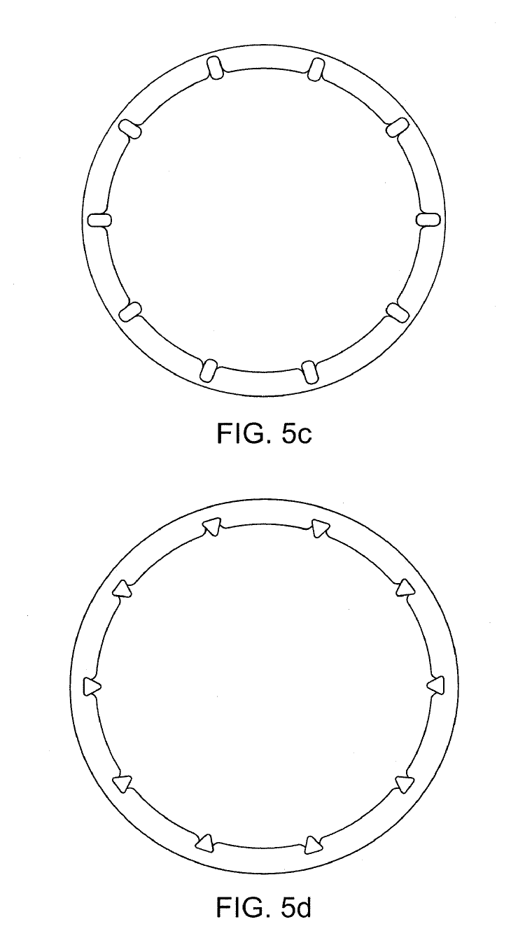

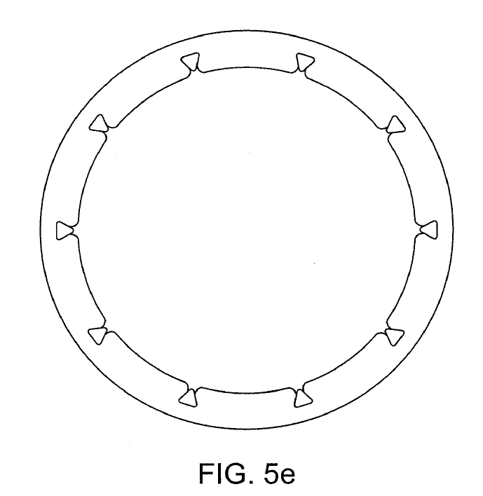

Design features can be added to the design shown in FIG. 3 to gain benefits of enabling embedding the struts into the vessel wall in addition to or in combination with reducing strut width. FIG. 4 illustrates various cross-sections of struts, including rectangular, triangular, pentagon/house, rounded protrusions, teardrop, asymmetrical triangle, square, and other variations.

The stent implant may be manufactured using various methods, such as chemical etching, chemical milling, laser cutting, stamping, EDM, waterjet cutting, bending of wire, injection molding, and welding. A surface modification may be applied to enhance stent coating retention and integrity upon expansion.

The raw material may start as wire, drawn tubing, co-drawn tubing for multiple layer stent constructions, flat sheet, or other forms. The raw material may be permanent, such as 316L stainless steel, cobalt-chromium alloy (L-605, MP35N), eligiloy, nitinol alloy, platinum, palladium, tantalum, or other alloys. Alternatively, biodegradable materials may also be used, such as magnesium alloys or PLLA polymers. The implant can be formed from metals including alloys, polymers, ceramics, or combinations thereof, such as stainless steel alloys, steel alloys, cobalt-chromium alloys, nickel-titanium alloys, platinum-iridium alloys, platinum enhanced alloys such as PERSS (Platinum Enhanced Radiopaque Stainless Steel), molybdenum-rhenium alloys such as NULOY™, magnesium alloys, Elgiloy, platinum, tantalum, titanium, iron, niobium, magnesium, palladium, PLLA, PLGA, cellulose, etc.

The present invention also applies to implants used for prosthetic valves or filters.

While the above is a complete description of the preferred embodiments of the invention, various alternatives, modifications, and equivalents may be used. Therefore, the above description should not be taken as limiting the scope of the invention which is defined by the appended claims.

1.-42. (canceled)

43. A vascular prosthesis comprising:
a metal scaffold including struts;
wherein the scaffold has a metal to artery ratio ranging from 1% to 12% when expanded to a diameter ranging from 2 mm to 4 mm; and
wherein the scaffold has at least one of (a) struts having a thickness in the range from 0.001 inch to 0.005 inch; (b) a crush strength in the range from 2 psi to 15 psi to crush at least 25% in diameter; and (c) an acute recoil below 15%.

44. A vascular prosthesis as in claim 43, wherein the prosthesis comprises a stent.

45. A vascular prosthesis as in claim 43, wherein the scaffold has an open cell design.

46. A vascular prosthesis as in claim 43, wherein the scaffold has a closed cell design.

47. A vascular prosthesis as in claim 43, wherein the metal scaffold comprises a material comprising at least one member selected from the group consisting of iron, magnesium, niobium, palladium, platinum, tantalum, titanium, cobalt-chromium alloys, L-605 cobalt-chromium alloy, MP35N cobalt-chromium alloy, Elgiloy®, magnesium alloys, molybdenum-rhenium alloys, NULOY™, nickel-titanium alloys, platinum-iridium alloys, platinum-enhanced alloys, PERSS (Platinum-Enhanced Radiopaque Stainless Steel), steel alloys, stainless steel alloys, and 316L stainless steel.

48. A vascular prosthesis as in claim 43, wherein the struts are arranged in a plurality of successive rings.

49. A vascular prosthesis as in claim 48, wherein the struts of at least some adjacent rings have same width.

50. A vascular prosthesis as in claim 48, wherein the struts of at least some adjacent rings have different width.

51. A vascular prosthesis as in claim 48, wherein the rings are axially joined by links.

52. A vascular prosthesis as in claim 48, wherein each of the plurality of successive rings includes from 2 crowns to 12 crowns and from 4 struts to 22 struts.

53. A vascular prosthesis as in claim 43, wherein the maximum total scaffold cross-sectional area ranges from 10 mil² to 100 mil².

54. A vascular prosthesis as in claim 43, wherein the total scaffold surface area ranges from 1 mil² to 3.7 mil², or from 1 mm² to 3.7 mm², per mm length of the scaffold at an expanded diameter.

55. A vascular prosthesis as in claim 43, wherein the total scaffold volume ranges from 0.008 mil³ to 0.065 mil³, or from 0.008 mm³ to 0.06 mm³, per mm length of the scaffold at an expanded diameter.

56. A vascular prosthesis as in claim 43, wherein said scaffold is balloon expandable.

57. A vascular prosthesis as in claim 56, wherein said scaffold has a crimped profile ranging from 0.01 to 0.045 inch.

58. A vascular prosthesis as in claim 43, wherein at least some of the struts have a cross-sectional geometry which enhances vessel wall penetration upon expansion.

59. A vascular prosthesis as in claim 43, wherein at least part of the scaffold is coated with at least one polymer selected from the group consisting of poly(methyl methacrylate), poly(2-hydroxyethyl methacrylate), poly(butyl methacrylate), poly(ethylene vinyl acetate), poly(ethylene-co-vinyl alcohol), poly(styrene-isobutylene styrene), poly(hexyl methacrylate-co-vinyl pyrrolidone-co-vinyl acetate), poly(N-vinyl-2-pyrrolidone), poly(butyl methacrylate-co-ethylene-co-vinyl acetate), poly(butyl methacrylate-co-vinyl acetate), fluoropolymers, polytetrafluoroethylene, polyvinylidene fluoride, polyvinylidene fluoride-co-hexafluoropropylene, polyethylene glycol, polydimethyl siloxane, polycarbonates, polyethylene carbonate, phosphorylcholine, aliphatic polyesters, polylactides, polyglycolic acid, polycaprolactone, poly(L-lactic acid-co-ε-caprolactone), polydioxanone, polyhydroxybutyrates, polyhydroxyvalerates, polyanhydrides, polyortho esters, polyether esters, polyiminocarbonates, polyester amides, and blends and copolymers of the above.

60. A vascular prosthesis as in claim 43, wherein at least part of the scaffold comprises a coating comprising at least one pharmacological agent selected from the group consisting of immunomodulator macrocyclic lactones, anti-proliferative agents, anti-cancer agents, anti-inflammatory agents, anti-thrombotic agents, anti-platelet agents, anti-diabetic agents, anti-hyperlipidemia agents, anti-hypertensive agents, anti-angiogenic agents, angiogenic agents, healing-promoting agents, and anti-fungal agents.

61. A vascular prosthesis as in claim 60, wherein the is at least one pharmacological agent is selected from the group consisting of rapamycin, everolimus, zotarolimus (ABT 578), AP20840, AP23841, AP23573, CCI-779, deuterated rapamycin, Novolimus, TAFA93, tacrolimus, TKB662, cyclosporine, taxol, dexamethasone, heparin, trapidil, and analogues, pro-drugs, metabolites, and salts thereof.

62.-106. (canceled)

107. A vascular prosthesis as in claim 43, wherein the scaffold has a metal to artery ratio ranging from 2% to 9% when expanded to a diameter ranging from 2 mm to 4 mm.

108. A vascular prosthesis as in claim 43, wherein the struts have a width in the range from 0.0004 inch to 0.0035 inch.

109. A vascular prosthesis as in claim 43, wherein the struts have a width that is less than the thickness of the struts.

110. A vascular prosthesis as in claim 109, wherein the thickness of the struts is at least 150% of the width of the struts.

111. A vascular prosthesis as in claim 43, wherein the scaffold has an outer surface area of less than 0.8 mm² per mm length of the scaffold at an expanded diameter.

112. A vascular prosthesis as in claim 43, wherein the scaffold has a maximum circular unsupported area of less than or equal to 2.0 mm diameter (3.14 mm² area) at an expanded diameter.
